(19) United States
(12) Patent Application Publication
(10) Pub. No.: US 2026/0283150 A1
(43) Pub. Date: Sep. 24, 2026

(54) SPORICIDAL SOLUTION AND USES THEREOF

(71) Applicant: Medivators Inc., Minneapolis, MN (US)

(72) Inventors: Huyen Bui, Brooklyn Park, MN (US); Nam Bui, Brooklyn Park, MN (US); Kristopher Murphy, New Brighton, MN (US); Tuan Nguyen, Chaska, MN (US); Erin Rodrigues, Youngsville, NC (US)

(21) Appl. No.: 19/471,867

(22) PCT Filed: Apr. 15, 2024
(86) PCT No.: PCT/US2024/024636

§ 371 Date: Oct. 2, 2025

Related U.S. Application Data
(60) Provisional application No. 63/497,380, filed on Apr. 20, 2023.

(51) Int. Cl. A01N 37/16; A01N 25/04; A01N 25/30; A01N 37/02; A01N 59/00; A01P 1/00

(52) U.S. Cl. CPC A01N 37/16; A01N 25/04; A01N 25/30; A01N 37/02; A01N 59/00; A01P 1/00

(57) ABSTRACT

Various embodiments disclosed relate to a sporicidal solution. The sporicidal solution includes less than about 7 wt % acetic acid, propionic acid, or a mixture thereof. The solution further includes about 0.22 wt % to about 10 wt % hydrogen peroxide. The solution further includes about 300 ppm to about 2000 ppm peracetic acid, perpropionic acid or both. The solution further includes about 0.0098 wt % to about 1.2 wt % stabilizer. The solution further optionally includes about 10 wt % to about 80 wt % isopropyl alcohol, less than or equal to 5 wt % formic acid, and/or 0.05 wt % to about 0.5 wt % surfactant.

### CLAIM OF PRIORITY AND CROSS-REFERENCE TO RELATED APPLICATIONS

This application claims the benefit of priority to U.S. Provisional Patent Application Ser. No. 63/497,380 entitled “SPORICIDAL SOLUTION AND USES THEREOF,” filed Apr. 20, 2023, the disclosure of which is incorporated herein in its entirety by reference.

### BACKGROUND

Various solutions can be used to disinfect a surface. However, despite a solution's ability to effectively disinfect, there can be drawbacks associated with the formulas including a solution's corrosive properties and propensity to leave a residue on the surface. It is desirable to develop disinfecting solutions that are able to disinfect a surface while minimizing undesirable characteristics.

### SUMMARY OF THE INVENTION

Various embodiments disclosed relate to a sporicidal solution. The sporicidal solution includes less than about 7 wt % formic acid, acetic acid, propionic acid, or a mixture thereof. The solution further includes about 0.4 wt % to about 10 wt % hydrogen peroxide. The solution further includes about 300 ppm to about 2000 ppm peracetic acid, perpropionic acid or both. The solution further includes about 0.01 wt % to about 1.2 wt % stabilizer. The solution further optionally includes about 10 wt % to about 80 wt % isopropyl alcohol. The solution further optionally includes a 0.05 wt % to about 0.5 wt % surfactant.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl, or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH), —C₂H₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃—, wherein each R is independently selected, and protonated forms of each, except for —NR₃′, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon. e.g., a hydrocarbon that is bonded at two locations.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

According to various aspects of the present disclosure, a sporicidal solution is described. The sporicidal solution is advantageous in that it is characterized as being “low odor” and “low residual”. That is the sporicidal solution does not produce undesirable odors during or following application to a surface. Additionally, following application, the sporicidal solution does not leave a significant amount of visible residue on the surface to which it is applied. Additionally, the components of the sporicidal solution are chosen to substantially prevent or at least reduce corrosion in the surface attributable to the sporicidal solution.

An example of a particular low-odor sporicidal solution is one that includes a stock solution of 4-6% PAA, 20-26% hydrogen peroxide, and 8-11% acetic acid. That stock solution can be diluted to include 0.3-0.5% hydrogen peroxide, 0.1-0.25% acetic acid, and 600-1000 ppm PAA

The surface to which the sporicidal solution is applied can include many suitable surfaces. For example, the surface can be at least a portion of a medical device, at least a portion of an industrial surface, at least a portion of a residential surface, or a combination thereof. An industrial surface can include a floor, wall, countertop, or the like of a disinfect room, hospital, clinic, factory, food-processing plant, or the like. A residential surface can include a floor, wall, countertop, or the like.

The function and benefits of the sporicidal solution are a function of the solution's ingredients. Those ingredients can include acetic acid, propionic acid, or a mixture thereof; hydrogen peroxide; peracetic acid, perpropionic acid, or both; a stabilizer; and optionally isopropyl alcohol.

The formic acid, acetic acid, propionic acid, or a mixture thereof can be less than about 7 wt % of the solution, less than about 5 wt %, in a range of from 0.1 wt % to 7 wt %, 2 wt % to 6 wt %, or 5 wt % to 7 wt %. The hydrogen peroxide can be present in an amount in a range of about 0.22 wt % to about 10 wt %, about 1 wt % to about 5 wt % hydrogen peroxide, less than, equal to, or greater than about 0.22 wt %, 0.5, 1, 1.5, 2, 2.5, 3, 3.5, 4, 4.5, 5, 5.5, 6, 6.5, 7, 7.5, 8, 8.5, 9, 9.5, or about 10 wt %. The peracetic acid, perpropionic acid or both can be present in an amount ranging from about 300 ppm to about 2000 ppm, about 400 ppm to about 1000 ppm, less than, equal to, or greater than about 300 ppm, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 ppm. The stabilizer can be in a range of from 0.0098 wt % to about 1.20 wt %, about 0.08 wt % to about 0.50 wt %, less than, equal to, or greater than about 0.0098 wt %, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 1.05, 1.10, 1.15, or about 1.20 wt %. Where present, the isopropyl alcohol can be in a range of from about 10 wt % to about 80 wt % of the solution, about 20 wt % to about 45 wt %, less than, equal to, or greater than about 10 wt %, 15, 20, 25, 30, 35, 40, 45, 50, 55, 60, 65, 70, 75, or about 80 wt %.

In examples where isopropyl alcohol is present, the isopropyl alcohol can be part of a single part solution that includes all of the components of the solution. In alternative examples, the isopropyl alcohol can be present in a second, distinct, part that not combined with the first part (including additional components) until the solution is ready for use. When present as a two part solution. A wt:wt ratio of the first part to the second part can be in a range of from about 50:1 to about 1:70, about 1:50 to about 1:70, about 3:1 to about 1:3, less than, equal to, or greater than about 5:1, 4:1, 3:1, 2:1, 1.1, 1.2, 1:3, 1:4, or 1:5.

Peracid solutions suitable for the present solution can be derived by oxidation of the corresponding aliphatic carboxylic acid with aqueous hydrogen peroxide, optionally in the presence of a strong acid catalyst, and often contain residual amounts of both the carboxylic acid and hydrogen peroxide.

In some examples, the stabilizer can be 1-hydroxyethylidene-1,1,-diphosphonic acid, 2,6-dipicolinic acid, or a mixture thereof. In further examples, the stabilizer can be a polymeric stabilizer that is selected from a phosphino polycarboxylic acid, or salt thereof. In some examples, the phosphino polycarboxylic acid has formula (I)

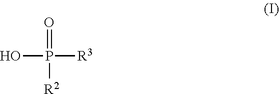

wherein R² is

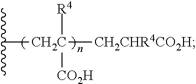

R³ is

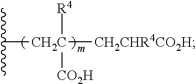

R⁴, at each occurrence, is independently hydrogen or C₁₋₄alkyl; and m and n are each independently an integer, where m+n is an integer from 30 to 60. In some examples, R⁴ is hydrogen. In some examples, the phosphino polycarboxylic acid has a molecular weight of 3300-3900 g/mol.

In some examples, the one or more polymeric stabilizers is selected from a polymer, or salt thereof, with molecular weight of 3000 to 15,000 g/mol, the polymer being derived from a plurality of monomer units of each of

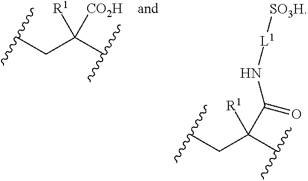

In some examples, the polymer is derived from a plurality of monomer units of each of

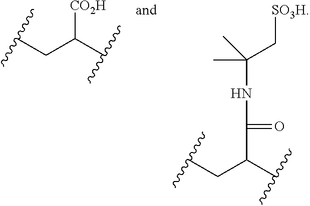

The polymeric stabilizers can include the specified monomer units.

In some examples, the one or more polymeric stabilizers is selected from a polymer, or salt thereof, with molecular weight of 3000 to 15,000 g/mol, the polymer being derived from a plurality of monomer units of each of

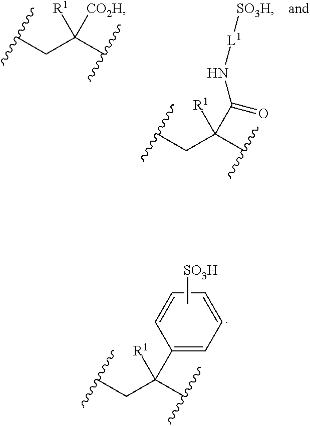

In some examples, the polymer is derived from a plurality of monomer units of each of

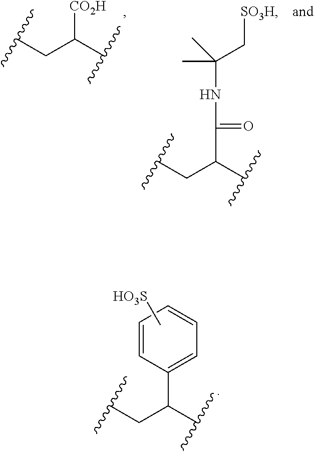

The polymeric stabilizers can include the specified monomer units.

In some examples, the peroxide solution is stabilized with 0.1-1500 ppm of the one or more polymeric stabilizers. The use of the polymeric stabilizer system herein does not preclude or restrict the presence of other known stabilizers for peroxygens. Stabilized peroxyacid solutions of the invention may include additional stabilizers or additives, such as a phosphate, a stannate, or a chelant. Compounds such as polycarboxylic acids, including for example dipicolinic acid, ethylenediaminetetraacetic acid or citric acid, soluble salts of phosphates which may take the form of simple monomeric species, or of condensed linear polyphosphates or cyclic metaphosphates. Further known stabilizers which may be included are organophosphonates, which molecules may additionally contain other functional groups such as hydroxy or amino. These are exemplified in compounds such as 1-hydroxyetliylidene-1,1-diphosphonic acid and poly(methyleneamino)-phosphonic acids such as aminotri(methylenephosphonic acid), and diethylenetriaminepenta(methylenephosphonic acid).

Useful stannates include an alkali metal stannate, particularly sodium stannate (Na₂(Sn(OH)₆). Stannates further include stannic chloride, stannic oxide, stannic bromide, stannic chromate, stannic iodide, stannic sulfide, tin dichloride bis(2,4-pentanedionate), tin phthalocyanine dichloride, tin acetate, tin t-butoxide, di-n-butyl tin(IV) dichloride, tin methacrylate, tin fluoride, tin bromide, stannic phosphide, stannous chloride, stannous fluoride, stannous pyrophosphate, sodium stannate, stannous 2-ethylhexoate, stannous bromide, stannous chromate, stannous fluoride, stannous methanesulfonate, stannous oxalate, stannous oxide, stannous sulfate, stannous sulfide, barium stannate, calcium stannate, copper(II) stannate, lead stannate dihydrate, zinc stannate, sodium stannate, potassium stannate trihydrate, strontium stannate, cobalt(II) stannate dihydrate, sodium trifluorostannate, ammonium hexachlorostannate, and lithium hexafluorostannate.

Another optional component of the stabilized peroxy acid solutions is the soluble salt of a phosphate. The phosphate salt can take the form of the simple monomeric species, or of the condensed linear polyphosphate, or cyclic polyphosphate(metaphosphate). The monomeric phosphate salts are of the general formula, MₙH_{q}PO₄, (in which q=0, 1, or 2; n=1, 2, or 3; n+q=3). Here M can be one or more monovalent cations selected from the following: Li, Na, K, NH₄, NR₄ (where R represents an alkyl chain containing 1 to 5 C atoms). The polyphosphates have the general formula, Mₙ₊₂PₙO₃ₙ₊₁ where n=2 to 8, and M can be chosen from Li, Na, K, NH₄, NR₄ where R represents an alkyl chain containing 1 to 5 C atoms). The cyclic polyphosphates have the general formula MₙPₙO₃ₙ where n=3 to 8 and M can be chosen from Li, Na, K, NH₄, NR₄ where R represents a linear or branched alkyl group containing 1 to 5 C atoms). The above may be optionally introduced into the stabilizer system in their acid form. Exemplary phosphates include pyrophosphoric acid and metaphorphoric acid and their salts, e.g., sodium salts.

Any phosphonic acid based chelant can optionally be used, such as amino trimethylene phosphonic acid (ATMP), 2-phosphonobutane-1,2,4-tricarboxylic acid (PBTCA), N-sulfonic amino dimethylene phosphonic acid (SADP), methylamine dimethylene phosphonic acid (MADMP), glycine dimethyl phosphonic acid (GDMP), 2-hydroxyphosphonocarboxylic acid (HPAA), polyhydric alcohol phosphate ester (PAPE), 1-hydroxyethylidene-1,1-diphosphonic acid (HEDP), 1-aminoethane-1,1-diphosphonic acid, amino tri(methylenephosphonic acid) (ATMP), ethylene diamine tetra(methylenephosphonic acid), hexamethylene diamine tetra(methylenephosphonic acid), diethylenetriamine penta(methylenephosphonic acid) (DTPMP), diethylenetriamine hexa(methylenephosphonic acid), and 1-aminoalkane-1,1-diphosphonic acids such as morpholinomethane diphosphonic acid. N,N-dimethyl aminodimethyl diphosphonic acid, aminomethyl diphosphonic acid, or salts thereof, for example sodium salts.

Yet further stabilizers to be contemplated are free radical scavengers. Useful radical scavengers include pyridine carboxylic acids, such as 2,6-pyridine dicarboxylic acid and picolinic acid. Scavengers may be selected from amongst phenols, polyols, or thiols. Suitable scavengers are those selected from phenols which satisfy the general formula (H)

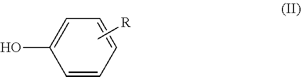

in which R represents at least one substituent selected from alkyl, ether, hydroxyl, carboxylic acid, and aliphatic carboxylic acid ester groups. Normally R in (II), represents from 1 to 4 substituents. One suitable sub-class of phenolic scavengers polyhydroxybenzoic acid or alkyl ester derivatives thereof, the benzene nucleus optionally being further substituted by one or more alkyl substituents. Normally not more than a single carboxylic acid/ester substituent is present. Included within that sub-class are the dihydroxybenzoic acids, gallic acid, pyrogallic acid and ester derivatives thereof. Other suitable sub-classes comprise polyhydroxy alkylbenzenes or ether derivatives thereof. Representatives of that subgroup include alkylresorcinols and alkylhydroquinones. Further suitable radical scavengers are substituted polyaryl compounds including those where one or more of the aryl rings is substituted with a heteroatom. A suitable example from this group is 8-hydroxyquinoline.

Also to be contemplated as phosphorus containing salts are organophosphonates which may be introduced as a soluble salt or as the parent acid. Compounds which may be contemplated include, ethylphosphonic acid, propylphosphonic acid, butylphosphonic acid, t-butylphosphonic acid, or phenylphosphonic acid. Additionally the phosphonic acid molecules can contain other functional groups such as hydroxy or amino. These are exemplified in compounds such; as 1-hydroxyethylidene-1,1-diphosphonic acid, and poly(methyleneamino) phosphonic acids such as aminotri(methylene phosphonic acid), and diethylenetriaminepenta(methylenephosphonic acid).

Further additives which may be contemplated are sulphuric acid, or soluble salts thereof. Organosulphonic acids such as decylsulphonic acid, dodecylsulphonic acid, toluenesulphonic acid and methylsulphonic acid, as well as their salts, may also be introduced. Stannate may be employed at a concentration of up to 15% of the total stabilizer solution, or at a concentration of between 8 to 12%.

It is to be understood that the solution of the disclosure can include mixtures of one or more polymeric stabilizers, two or more of at least one protective colloid at least one radical scavenger and at least one phosphonic acid based chelant.

Accordingly, in some examples of the present disclosure there is provided a process for the stabilization of an aqueous solution of a peroxyacid by means of the addition of an effective amount of one or more polymeric stabilizer thereto.

In specific examples, the solution of the present disclosure can be stable over extended periods of time (i.e., has a long-term stability). The term “long-term stability” refers to a substance undergoing little or no physical and/or chemical desolation or degradation, over extended periods of time. In further specific examples, the solution of the present disclosure can be stable over extended periods of time, such that at about 1 atm and about 19° C., less than about 5 wt. % of each component independently degrades over about one year. In additional specific examples, the solution of the present disclosure can be stable over extended periods of time, such that at about 1 atm and about 19° C., at least about 95 wt. % of each component is independently present after about one year. Having the solution be relatively stable over extended periods of time will allow the solution to retain its effectiveness over that time, ensuring that it will remain useful and active for its intended purpose. In contrast, in those solutions that do not retain their effectiveness over that time, product loss can result, which can be financially costly.

The solution of the present disclosure can be used to effectively reduce the number of microbes located upon a substrate. In specific examples, the solution can effectively kill and/or inhibit a microorganism (e.g., virus, fungus, mold, slime mold, algae, yeast, mushroom and/or bacterium), thereby disinfecting the substrate. While not so limited examples of microbes the solution can effectively kill (e.g., >4 log or >6 log reduction) include Chaetomium globosum, S. aureas, P. Aeruginosa, T. interdigitale, B. subtilis, poliovirus, and minute virous of mouse.

In some examples adding formic acid in a concentration ranging from 0.2 to about 2 wt % can increase the sporicidal effectiveness of the solution especially against Chaetomium globosum.

In additional specific examples, the solution can effectively sanitize a substrate, thereby simultaneously disinfecting and disinfecting the substrate. In additional specific examples, the solution can effectively kill or inhibit all forms of life, not just microorganisms, thereby acting as a biocide.

Additionally, as stated hereinabove a particular benefit of the solution is that it has low corrosion characteristics. For example, the solution may have a corrosion rate of brass in a range of from about 0.01 mg/min to about 1 mg/min, about 0.04 mg/min to about 0.915 mg/min, less than, equal to, or greater than about 0.01 mg/min, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or about 1 mg/min.

Additionally, as stated hereinabove, a very small amount of the solution remains on the surface/substrate after application. For example, following application about 0.01 wt % to about 0.50 wt % of the total amount of solution applied remains, about 0.05 wt %/o to about 0.80 wt %, less than, equal to, or greater than about 0.05 wt %, 0, 10, 0.15, 0.20, 0.25, 0.30, 0.35, 0.40, 0.45, 0.50, 0.55, 0.60, 0.65, 0.70, 0.75, or about 0.80 wt %.

In specific examples, the solution can effectively disinfectant a substrate. In further specific examples, the solution can effectively disinfectant the surface of a substrate. In additional specific examples, the solution can effectively sterilize a substrate. In further specific examples, the solution can effectively sterilize the surface of a substrate.

The solution of the present invention can be formulated for application, depending upon the users preference as well as the ultimate application of the solution. For example, the solution can be formulated for use in a sprayable solution, atomized liquid sprayer, or liquid applicator. Such formulations can include at least one of a spray bottle, motorized sprayer, wipe, cloth, sponge, non-woven fabric, and woven fabric. Such formulations may be particularly suitable for applying the solution to a surface of a hospital, physician's office, medical clinic, medical facility, dental office, dental facility, airport, school, pet store, zoo, children's day care, elderly nursing home, museum, movie theatre, athletic facility, sporting arena, gymnasium, rest room, bathroom, shopping center, amusement park, church, synagogue, mosque, temple, restaurant, food processing facility, food manufacturing facility, pharmaceutical company, hot-tub, sauna, and/or disinfect room. Such liquid formulations may be particularly suitable for applying the solution to metal, plastic, natural rubber, synthetic rubber, glass, stone, grout, fiberglass, wood, concrete, construction products, and/or building products.

Packaging materials (e.g., for food) may be disinfected with the stabilized peroxyacid solutions described herein. In some examples, the packaging material is made of PET. In some examples, the packaging material is contacted with the stabilized peroxyacid solution for sufficient time to disinfect the packaging material. In some examples, the process is a rinser cold disinfection, which rinses bottles (upside down) with an aqueous disinfectant solution, mostly based on a peroxy acid (e.g., peracetic acid) as active chemical. A second washing step with clear water must follow to remove remaining disinfectant. The rinser approach does not need high temperatures and is also named cold aseptic. Average consumption of peracetic acid is about 0.2-0.5 ml per unit.

In specific examples, the solution of the present invention can be non-toxic. The term “non-toxic” refers to a substance that has a relatively low degree to which it can damage a living or non-living organism. Toxicity can refer to the effect on a whole organism, such as an animal, bacterium, or plant, as well as the effect on a substructure of the organism, such as a cell (cytotoxicity) or an organ (organotoxicity), such as the liver (hepatotoxicity). A central concept of toxicology is that effects are dose-dependent; even water can lead to water intoxication when taken in large enough doses, whereas for even a very toxic substance such as snake venom there is a dose below which there is no detectable toxic effect. Having the solution be relatively non-toxic will allow a wider range of users be able to safely handle the solution, without serious safety concerns or risks.

The present invention also provides for a kit that includes: (a) an enclosed container that includes a removable closure; (b) the solution of the present invention as described herein, which is located inside the enclosed container; and (c) printed indicia located on the enclosed container.

In specific examples, the enclosed container can be opaque. In additional specific examples, the enclosed container can be manufactured from high density polyethylene (HDPE), thereby providing the requisite opacity. Having the enclosed container be manufactured from high density polyethylene (HDPE) will decrease the likelihood that the solution will degrade and/or decompose over extended periods of time, due to excessive exposure to direct sunlight.

### Examples

Various embodiments of the present invention can be better understood by reference to the following Examples which are offered by way of illustration. The present invention is not limited to the Examples given herein.

The Tables below present data relating to the properties and microbiological properties of various solutions.

TABLE 1

| | | | Current | | | | | | |
|---|---|---|---|---|---|---|---|---|---|
| | | Production | Prelim | | | Corrosion | | | Surface |
| Formulation | Active | Process | Stability | | | Brass | % | Residual | Tension |
| # | components | Time | (Days) | pH | Density | mg/min | Residual | Visual | (mN/m) |
| 1 | 3% H₂O₂, 580 ppm | 75 min | 378 | 2.29 | 1.01 | 0.915 | 0.05 | Clear | 62.97 |
| | PAA, 200 ppm | | | | | | | | |
| | PPA | | | | | | | | |
| 2 | 3% H₂O₂, 200 ppm | 80 min | 378 | 2.29 | 1.01 | 0.342 | 0.08 | Clear | 56.34 |
| | PAA, 580 ppm | | | | | | | | |
| | PPA | | | | | | | | |
| 3 | 3% H₂O₂, | 75 min | 378 | 2.29 | 1.01 | 0.739 | 0.08 | Clear | 65.30 |
| | 580 ppmPAA | | | | | | | | |
| 4 | 3% H₂O₂, 580 ppm | 80 min | 378 | 2.30 | 1.01 | 0.325 | 0.08 | Clear | 58.37 |
| | PPA | | | | | | | | |
| 5 | 5% H₂O₂, 750 ppm | 65 min | 378 | 2.00 | 1.02 | 0.527 | 0.21 | Very small | 66.97 |
| | PAA | | | | | | | white spots | |
| 6 | 5% H₂O₂, 750 ppm | 75 min | 378 | 2.00 | 1.02 | 0.474 | 0.21 | Very small | 60.72 |
| | PPA | | | | | | | white spots | |
| 7 | 5% H₂O₂, 400 ppm | | 266 | 2.04 | 1.01 | 0.545 | 0.25 | Very small | 63.64 |
| | PAA, 400 ppm | | | | | | | white spots | |
| | PPA | | | | | | | | |
| 8 | 5% H₂O₂, 540 ppm | | 266 | 2.03 | 1.02 | 0.711 | 0.23 | Very small | 62.26 |
| | PAA, 540 ppm | | | | | | | white spots | |
| | PPA | | | | | | | | |
| 9 | 5% H₂O₂, 750 | 65 min | 211 | 2.04 | 1.01 | 0.574 | 0.19 | Small oily | 29.07 |
| | ppmPAA | | | | | | | spots | |
| 10 | 5% H₂O₂, 750 ppm | 75 min | 211 | 2.05 | 1.01 | 0.390 | 0.32 | Small oily | 28.89 |
| | PPA | | | | | | | spots | |
| 11 | 5% H₂O₂, 750 ppm | 65 min | 203 | 2.14 | 1.01 | 0.340 | 0.42 | White powder. | 32.96 |
| | PAA | | | | | | | Very noticeable | |
| 12 | 5% H₂O₂, 750 ppm | 75 min | 203 | 2.15 | 1.01 | 0.241 | 0.45 | White powder. | 32.11 |
| | PPA | | | | | | | Very noticeable | |
| 13 | 5% H₂O₂, 750 ppm | 65 min | | 2.17 | 1.02 | 0.561 | 0.28 | Small oily | 58.13 |
| | PAA | | | | | | | spots | |
| 14 | 5% H₂O₂, 750 ppm | 75 min | | 2.18 | 1.02 | 0.345 | 0.27 | Small oily | 52.41 |
| | PPA | | | | | | | spots | |
| 1.1 | 2.1% H₂O₂, | 75 min | | 2.47 | 0.98 | 0.485 | 0.06 | Clear | 33.48 |
| | 406 ppm PAA, | | | | | | | | |
| | 140 ppm PPA | | | | | | | | |
| 1.2 | 2.1% H₂O₂, | 80 min | | 2.52 | 0.99 | 0.098 | 0.03 | Clear | 33.67 |
| | 140 ppm PAA, | | | | | | | | |
| | 406 ppm PPA | | | | | | | | |
| Comparative | 1% H₂O₂, 800 ppm | 2 days (1.5 | 1 year | 2.15 | 1.01 | 1.052- | 0.35 | Very small | 59.74 |
| | PAA | days for | | | | 1.109 | | white spots | |
| | | Renalin | | | | | | | |
| | | brew) | | | | | | | |

TABLE 2
Table 2

| | | Microbiological Data | | | | | | | |
|---|---|---|---|---|---|---|---|---|---|
| | | Bactericidal | Bactericidal | | Fungicidal/ | | | Virucidal Non- | |
| | | Cold Room | P. | P. | Yeasticidal | Sporicidal | | enveloped | |
| | | S. aureas | aeruginosa | aeruginosa | T. interdigitale | B. subtilis | B. subtilis | | Minute |
| | | ATCC 6538 | ATCC 15442 | ATCC 15442 | ATCC 9533 | ATCC 19659 | ATCC 6633 | Poliovirus | Virus of |
| | | 30 min 5 C. | 2 min RT | 5 min RT | 5 min 20 C. | 10 min 20 C. | 10 min 20 C. | Type 1 | Mouse |
| | | 5% Load | 5% Load | 5% Load | 5% Load Sus- | 5% Load Sus- | 5% Load Sus- | Dirty | 10 min 20 C. |
| Formu- | Active | Carrier Test | Carrier Test | Carrier Test | pension Test | pension Test | pension Test | Condition | Dirty |
| lation | compo- | (>6 Log | (>6 Log | (>6 Log | (>6 Log | (>6 Log | (>6 Log | (>4 Log | (>3 Log |
| # | nents | Reduction) | Reduction) | Reduction) | Reduction) | Reduction) | Reduction) | Reduction) | Reduction) |
| 1 | 3% H₂O₂, | Passed | Failed | Passed | Passed | Passed | Passed | Passed | Passed |
| | 580 ppm | | | | | (at 90%) | | (15 min) | |
| | PAA, | | | | | | | | |
| | 200 ppm | | | | | | | | |
| | PPA | | | | | | | | |
| 2 | 3% H₂O₂, | Passed | Failed | Passed | Passed | Passed | Passed | Passed | Passed |
| | 200 ppm | | | | | (at 90%) | | (30 min) | |
| | PAA, | | | | | | | | |
| | 580 ppm | | | | | | | | |
| | PPA | | | | | | | | |
| 3 | 3% H₂O₂, | | | | Passed | | Passed | | |
| | 580 | | | | | | | | |
| | ppmPAA | | | | | | | | |
| 4 | 3% H₂O₂, | | | | Passed | | Passed | | |
| | 580 ppm | | | | | | | | |
| | PPA | | | | | | | | |
| 5 | 5% H₂O₂, | Passed | Failed | Failed | Passed | Passed | Passed | Passed | Passed |
| | 750 ppm | | | | | (at 90%) | | (15 min) | |
| | PAA | | | | | | | | |
| 6 | 5% H₂O₂, | Passed | Failed | Passed | Passed | Passed | Passed | Passed | Passed |
| | 750 ppm | | | | | (at 70%) | | (30 min) | |
| | PPA | | | | | | | | |
| 7 | 5% H₂O₂, | | | | | Passed | | | |
| | 400 ppm | | | | | (at 70%) | | | |
| | PAA, | | | | | | | | |
| | 400 ppm | | | | | | | | |
| | PPA | | | | | | | | |
| 8 | 5% H₂O₂, | | | | | Passed | | | |
| | 540 ppm | | | | | (at 60%) | | | |
| | PAA, | | | | | | | | |
| | 540 ppm | | | | | | | | |
| | PPA | | | | | | | | |
| 9 | 5% H₂O₂, | | | | | Passed | | | |
| | 750 ppm | | | | | (at 70%) | | | |
| | PAA | | | | | | | | |
| 10 | 5% H₂O₂, | | Failed | Passed | | Passed | | | |
| | 750 ppm | | | | | (at 60%) | | | |
| | PPA | | | | | | | | |
| 11 | 5% H₂O₂, | | | | | Passed | | | |
| | 750 ppm | | | | | (at 70%) | | | |
| | PAA | | | | | | | | |
| 12 | 5% H₂O₂, | | Passed | Passed | | Passed | | | |
| | 750 ppm | | | | | at 70%) | | | |
| | PPA | | | | | | | | |
| 13 | 5% H₂O₂, | | | | | Passed | | | |
| | 750 ppm | | | | | (at 70%) | | | |
| | PAA | | | | | | | | |
| 14 | 5% H₂O₂, | | | | | Passed | | | |
| | 750 ppm | | | | | at 70%) | | | |
| | PPA | | | | | | | | |
| 1.1 | 2.1% | | | | | Passed | | | |
| | H₂O₂, | | | | | | | | |
| | 406 ppm | | | | | | | | |
| | PAA, | | | | | | | | |
| | 140 ppm | | | | | | | | |
| | PPA | | | | | | | | |
| 1.2 | 2.1% | | | | | Passed | | | |
| | H₂O₂, | | | | | | | | |
| | 140 ppm | | | | | | | | |
| | PAA, | | | | | | | | |
| | 406 ppm | | | | | | | | |
| | PPA | | | | | | | | |

| | | | | | | | | |
|---|---|---|---|---|---|---|---|---|
| Com- | 1% H₂O₂, | Passed | Passed | Failed | Failed | Failed | Claim | Claim |
| patitor | 800 ppm | | (at 10 min | (Passed | (Passed | (Passed | 30 min | 25 min |
| | PAA | | contact time) | at 10 min | at 30 min | at 30 min | 20 C. | 20 C. |

| | | |
|---|---|---|
| 20 C. 5% | 20 C. 5% | 20 C. 5% |
| Load) | Load) | Load) |

Table 3 shows various formulations using 2,6-pyridiniedicarboxylic acid to stabilize the peracid chemistry.

TABLE 3

| | | | | | Stability Study at 54° C. for 2 weeks (EPA Guideline | | | |
|---|---|---|---|---|---|---|---|---|
| | | | | | for Accelerated Stability to Represent 2 Years) | | | |
| | | | | | Before | After 2 | Before | After 2 |
| Formula | Peracid | | Residual | % | Data | weeks Data | Data | weeks Data |
| # | Base | Stabilizer | (Visual) | Residual | (% H2O2) | (% H2O2) | (PAA (ppm)) | (PAA (ppm)) |
| 5 | PAA | Stabilizer 1 | Oily layer | 0.21 | 4.93 | 4.92 | 760 | 722 |
| 19 | PAA | Stabilizer 2 | Noticeable | 0.13 | 4.93 | 5.04 | 754 | 730 |
| | | | white crystal | | | | | |
| 18 | PAA | Stabilizer 3 | Some white | 0.08 | 4.92 | 5.24 | 745 | 746 |
| | | | crystal | | | | | |
| 23 | PAA | Stabilizer 4 | Small white | Not | 4.94 | 5.03 | 665 | 680 |
| | | | crystal | detectable | | | | |
| 24 | PAA | Stabilizer 5 | Small white | Not | 4.93 | 5.12 | 671 | 677 |
| | | | crystal | detectable | | | | |

Table 4 shows corrosion rate-s of brass when exposed to various compositions.

TABLE 4

| | Corrosion |
|---|---|
| Composition | Rate (mg/min) |
| SKRTU (1% H₂O₂, 800 ppm PAA | 1.109 |
| and 5% acetic acid) | |
| LOS 5 (5% H₂O₂, 700-800 ppm, | 0.527 |
| PAA, 1.5% acetic acid, 0.3% | |
| stabilizer) | |
| LOS 5 + 0.5% Formic Acid | 0.555 |
| LOS 5 + 1% Formic Acid | 0.722 |
| 1000 ppm PAA | 0.136 |
| 1000 ppm PAA + 1% Formic Acid | 0.215 |

Table 5 shows the residuals of various compositions.

TABLE 5

| | | % Dequest (a | |
|---|---|---|---|
| | | solution of 60% | |
| | | etidronic acid | |
| | | with the balance | |
| | | being water and a | |
| Sample | PAA | small amount of | Calculated |
| name | concentration | acetic acid) | % Residual |
| Sample 1 | 700 ppm | 0.014 | 0.008 |
| Sample 2 | 800 ppm | 0.017 | 0.010 |
| Sample 3 | 1000 ppm | 0.020 | 0.012 |
| LOS 5 | 750 | 0.30 | 0.210 |

Table 6 shows the sporicidal effect of various compositions against C. sporogenes ATCC 3584. In the study two sets of 10 tubes including C. sporogenes were exposed to the composition for 30 minutes with the fraction of tubes having a positive among of C. sporogenes noted. The first set of tubes were kept at a temperature of 1° C. and the second set of tubes were kept at 2° C. As shown both compositions had good sporicidal properties.

TABLE 6

| | |
|---|---|
| Contact | Positive/Total |

| Composition | Time | 1° C. Tube | 2° C. Tube |
|---|---|---|---|
| 700 ppm PAA | 30 minutes | 0/10 | 0/10 |
| 700 ppm PAA + | 30 minutes | 0/10 | 0/10 |
| 1% formic acid | | | |
| 635 ppm PAA + | 30 minutes | 0/10 | 0/10 |
| 1% formic acid | | | |
| 513 ppm PAA + | 30 minutes | 0/10 | 0/10 |
| 1% formic acid | | | |

Table 7 shows the average log reduction of C. globosum for various compositions.

TABLE 7

| | Average log reduction |
|---|---|
| Composition | of C. globosum |
| 2 wt % formic acid + LOS 5 | 4.21 |
| 1 wt % formic acid + LOS 5 | 3.29 |
| 0.5 wt % formic acid + LOS 5 | 1.02 |
| 0.2 wt % formic acid + LOS 5 | 0.14 |
| LOS 5 | −0.07 |

As shown, the data in Table 2 suggests that using 2,6-dipicolinic acid can also stabilize the formulations and maintain the concentration of the actives within the allowable tolerance for 2 weeks at 54° C. The data passed the EPA condition for the accelerated stability study.

The formulas shown in Tables 1-3 can include other components such as stabilizers, chelators, emulsifiers, or the like. For simplicity Tables 1-3 show the quantity of “active” components.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Aspect 1 provides a sporicidal solution, comprising:
less than about 7 wt % acetic acid, propionic acid, or a mixture thereof,
about 0.22 wt % to about 10 wt % hydrogen peroxide;
about 300 ppm to about 2000 ppm peracetic acid, perpropionic acid or both;
about 0.0098 wt % to about 1.2 wt % stabilizer;
about 0.05 wt % to about 0.5 wt % surfactant; and
optionally, about 10 wt % to about 80 wt % isopropyl alcohol.

Aspect 2 provides the sporicidal solution of Aspect 1, wherein the sporicidal solution comprises less than or equal to about 5 wt % acetic acid, propionic acid or both.

Aspect 3 provides the sporicidal solution of any one of Aspects 1 or 2, wherein the sporicidal solution comprises about 0.5 wt % to about 4.9 wt % formic acid, acetic acid, propionic acid, or both.

Aspect 4 provides the sporicidal solution of any one of Aspects 1-3, wherein the sporicidal solution comprises about 0.22 wt % to about 5 wt % hydrogen peroxide.

Aspect 5 provides the sporicidal solution of any one of Aspects 1-4, wherein the sporicidal solution comprises about 400 ppm to about 1000 ppm peracetic acid, perpropionic acid or both.

Aspect 6 provides the sporicidal solution of any one of Aspects 1-5, wherein the sporicidal solution comprises a mixture of peracetic acid and perpropionic acid.

Aspect 7 provides the sporicidal solution of any one of Aspects 1-6, wherein the sporicidal solution comprises about 0.0098 wt % to about 1.2 wt % stabilizer.

Aspect 8 provides the sporicidal solution of any one of Aspects 1-7, further comprising a cationic surfactant, an anionic surfactant, or a mixture thereof.

Aspect 9 provides the sporicidal solution of Aspect 8, wherein the surfactant comprises 1-hydroxyethylidene-1,1,-diphosphonic acid, ethylene oxide, sodium dodecyl sulfate, or a mixture thereof.

Aspect 10 provides the sporicidal solution of any one of Aspects 1-9, wherein the stabilizer comprises 1-hydroxyethylidene-1,1,-diphosphonic acid, 2,6-dipicolinic acid, or a mixture thereof.

Aspect 11 provides the sporicidal solution of any one of Aspects 1-10, wherein the sporicidal solution comprises about 20 wt % to about 45 wt % isopropyl alcohol.

Aspect 12 provides the sporicidal solution of any one of Aspects 1-11, wherein the sporicidal solution comprises the isopropyl alcohol.

Aspect 13 provides the sporicidal solution of any one of Aspects 1-12, wherein the sporicidal solution is free of the isopropyl alcohol.

Aspect 14 provides a sporicidal solution comprising:
a first part comprising:
   less than about 7 wt % acetic acid, propionic acid, or a mixture thereof;
   about 0.22 wt % to about 10 wt % hydrogen peroxide;
   about 300 ppm to about 2000 ppm peracetic acid, perpropionic acid or both;
   about 0.0098 wt % to about 0.5 wt % stabilizer; and
a second part separate from the first part, the second part comprising:
   about 60 wt % to about 80 wt % isopropyl alcohol.

Aspect 15 provides the sporicidal solution of Aspect 14, wherein the sporicidal solution comprises less than or equal to about 5 wt % acetic acid, propionic acid or both.

Aspect 16 provides the sporicidal solution of any one of Aspects 14 or 15, wherein the sporicidal solution comprises about 0.5 wt % to about 4.9 wt % formic acid, acetic acid, propionic acid or both.

Aspect 17 provides the sporicidal solution of any one of Aspects 14-16, wherein the sporicidal solution comprises about 0.22 wt % to about 5 wt % hydrogen peroxide.

Aspect 18 provides the sporicidal solution of any one of Aspects 14-17, wherein the sporicidal solution comprises about 400 ppm to about 1000 ppm peracetic acid, perpropionic acid or both.

Aspect 19 provides the sporicidal solution of any one of Aspects 14-18, wherein the sporicidal solution comprises a mixture of peracetic acid and perpropionic acid.

Aspect 20 provides the sporicidal solution of any one of Aspects 14-19, wherein the sporicidal solution comprises about 0.0098 wt % to about 1.2 wt % stabilizer.

Aspect 21 provides the sporicidal solution of any one of Aspects 14-20, further comprising a cationic surfactant, an anionic surfactant, or a mixture thereof.

Aspect 22 provides the sporicidal solution of Aspect 21, wherein the surfactant comprises 1-hydroxyethylidene-1,1,-diphosphonic acid, ethylene oxide, sodium dodecyl sulfate, or a mixture thereof.

Aspect 23 provides the sporicidal solution of any one of Aspects 14-22, wherein the stabilizer comprises 1-hydroxyethylidene-1,1,-diphosphonic acid, 2,6-dipicolinic acid, or a mixture thereof.

Aspect 24 provides the sporicidal solution of any one of Aspects 14-23, wherein the sporicidal solution comprises about 10 wt % to about 45 wt % isopropyl alcohol.

Aspect 25 provides the sporicidal solution of any one of Aspects 14-24, wherein the first part and the second part are combined at a ratio in a range of from about 1:50 to about 1:70 (first part:second part).

Aspect 26 provides the sporicidal solution of any one of Aspects 14-25, wherein the first part and the second part are combined at a ratio of about 1:1 (first part:second part).

Aspect 27 provides the sporicidal solution of any one of Aspects 14-26, wherein prior to combination, the first part and the second part are stored in separate containers.

Aspect 28 provides the sporicidal solution of any one of Aspects 14-27, wherein
the first part comprises:
   about 6 wt % to about 7 wt % acetic acid;
   about 0.22 wt % to about 1.5 wt % hydrogen peroxide;
   about 500 ppm to about 1000 ppm peracetic acid;
   about 0.0098 wt % to about 0.8 wt % 1-hydroxyethylidene-1,1,-diphosphonic acid; and
a second part comprises:
   about 60 wt % to about 80 wt % isopropyl alcohol.

Aspect 29 provides a sporicidal solution, comprising: less than about 7 wt % acetic acid, propionic acid, or a mixture thereof;
about 0.22 wt % to about 10 wt % hydrogen peroxide,
about 300 ppm to about 2000 ppm peracetic acid, perpropionic acid or both;
about 0.0098 wt % to about 0.5 wt % stabilizer

Aspect 30 provides the sporicidal solution of Aspect 29, wherein the sporicidal solution comprises less than or equal to about 5 wt % acetic acid.

Aspect 31 provides the sporicidal solution of any one of Aspects 29 or 30, wherein the sporicidal solution comprises about 0.5 wt % to about 4.9 wt % formic acid, acetic acid, propionic acid or both.

Aspect 32 provides the sporicidal solution of any one of Aspects 29-31, wherein the sporicidal solution comprises about 1 wt % to about 5 wt % hydrogen peroxide.

Aspect 33 provides the sporicidal solution of any one of Aspects 29-32, wherein the sporicidal solution comprises about 400 ppm to about 1000 ppm peracetic acid, perpropionic acid or both.

Aspect 34 provides the sporicidal solution of any one of Aspects 29-33, wherein the sporicidal solution comprises a mixture of peracetic acid and perpropionic acid.

Aspect 35 provides the sporicidal solution of any one of Aspects 29-34, wherein the sporicidal solution comprises about 0.0098 wt % to about 1.2 wt % stabilizer.

Aspect 36 provides the sporicidal solution of any one of Aspects 29-35, further comprising a cationic surfactant, an anionic surfactant, or a mixture thereof.

Aspect 37 provides the sporicidal solution of Aspect 36, wherein the surfactant comprises 1-hydroxyethylidene-1,1,-diphosphonic acid, ethylene oxide, sodium dodecyl sulfate, or a mixture thereof.

Aspect 38 provides the sporicidal solution of any one of Aspects 29-37, wherein the stabilizer comprises 1-hydroxyethylidene-1,1,-diphosphonic acid, 2,6-dipicolinic acid, or a mixture thereof.

Aspect 39 provides the sporicidal solution of any one of Aspects 29-38, comprising:
about 1 wt % to about 3 wt % acetic acid, propionic acid, or a mixture thereof;
about 3 wt % to about 10 wt % hydrogen peroxide;
about 400 ppm to about 1700 ppm peracetic acid, perpropionic acid or both;
about 0.1 wt % to about 0.3 wt % 1-hydroxyethylidene-1,1,-diphosphonic acid.

Aspect 40 provides a method of making the sporicidal solution of any one of Aspects 1-39, the method comprising:
combining the acetic acid, propionic acid, or a mixture thereof; the hydrogen peroxide; the peracetic acid, perpropionic acid or both; the stabilizer; and optionally, isopropyl alcohol.

Aspect 41 provides a method of using the sporicidal solution of any one of Aspects 1-40, the method comprising contacting the sporicidal solution with a substrate.

Aspect 42 provides the method of Aspect 41, wherein the substrate comprises at least a portion of a medical device, at least a portion of an industrial surface, at least a portion of a residential surface, or a combination thereof.

1-42. (canceled)

43. A sporicidal solution, comprising:
less than about 7 wt % acetic acid, propionic acid, or a mixture thereof;
about 0.22 wt % to about 10 wt % hydrogen peroxide;
about 300 ppm to about 2000 ppm peracetic acid, perpropionic acid or both;
about 0.0098 wt % to about 1.2 wt % stabilizer;
about 0.05 wt % to about 0.5 wt % surfactant; and
optionally, about 10 wt % to about 80 wt % isopropyl alcohol.

44. The sporicidal solution of claim 43, further comprising a cationic surfactant, an anionic surfactant, or a mixture thereof.

45. The sporicidal solution of claim 44, wherein the surfactant comprises 1-hydroxyethylidene-1,1,-diphosphonic acid, ethylene oxide, sodium dodecyl sulfate, or a mixture thereof.

46. The sporicidal solution of claim 43, wherein the stabilizer comprises 1-hydroxyethylidene-1,1,-diphosphonic acid, 2,6-dipicolinic acid, or a mixture thereof.

47. A sporicidal solution comprising:
a first part comprising:
less than about 7 wt % acetic acid, propionic acid, or a mixture thereof;
about 0.0.22 wt % to about 10 wt % hydrogen peroxide;
about 300 ppm to about 2000 ppm peracetic acid, perpropionic acid or both;
about 0.0098 wt % to about 0.5 wt % stabilizer; and
a second part separate from the first part, the second part comprising:
about 60 wt % to about 80 wt % isopropyl alcohol; and
about 0.05 wt % to about 0.5 wt % surfactant.

48. The sporicidal solution of any one of claim 47, wherein the sporicidal solution comprises about 400 ppm to about 1000 ppm peracetic acid, perpropionic acid or both.

49. The sporicidal solution of any one of claim 47, wherein the sporicidal solution comprises a mixture of peracetic acid and perpropionic acid.

50. The sporicidal solution of claim 47, further comprising a cationic surfactant, an anionic surfactant, or a mixture thereof.

51. The sporicidal solution of claim 50, wherein the surfactant comprises 1-hydroxyethylidene-1,1,-diphosphonic acid, ethylene oxide, sodium dodecyl sulfate, or a mixture thereof.

52. The sporicidal solution of claim 47, wherein the stabilizer comprises 1-hydroxyethylidene-1,1,-diphosphonic acid, 2,6-dipicolinic acid, or a mixture thereof.

53. The sporicidal solution of claim 47, wherein the sporicidal solution comprises about 10 wt % to about 45 wt % isopropyl alcohol.

54. The sporicidal solution of claim 47, wherein the first part and the second part are combined at a ratio in a range of from about 1:50 to about 1:70 (first part:second part).

55. The sporicidal solution of claim 47, wherein prior to combination, the first part and the second part are stored in separate containers.

56. The sporicidal solution of any claim 47, wherein
the first part comprises:
about 6 wt % to about 7 wt % acetic acid;
about 0.5 wt % to about 1.5 wt % hydrogen peroxide;
about 500 ppm to about 1000 ppm peracetic acid;
about 0.3 wt % to about 0.8 wt % I-hydroxyethylidene-1,1,-diphosphonic acid; and
a second part comprises:
about 60 wt % to about 80 wt % isopropyl alcohol; and
about 0.05 wt % to about 0.5 wt % surfactant.

57. A sporicidal solution, comprising:
less than about 7 wt % formic acid, acetic acid, propionic acid, or a mixture thereof;
about 0.22 wt % to about 10 wt % hydrogen peroxide;
about 300 ppm to about 2000 ppm peracetic acid, perpropionic acid or both;
about 0.0098 wt % to about 0.5 wt % stabilizer.

58. The sporicidal solution of claim 57, wherein the sporicidal solution comprises less than or equal to about 5 wt % formic acid, acetic acid, propionic acid, or mixture thereof.

59. The sporicidal solution of claim 57, further comprising a cationic surfactant, an anionic surfactant, or a mixture thereof.

60. The sporicidal solution of claim 59, wherein the surfactant comprises 1-hydroxyethylidene-1,1,-diphosphonic acid, ethylene oxide, sodium dodecyl sulfate, or a mixture thereof.

61. The sporicidal solution of claim 57, wherein the stabilizer comprises 1-hydroxyethylidene-1,1,-diphosphonic acid, 2,6-dipicolinic acid, or a mixture thereof.

62. The sporicidal solution of claim 57, comprising:
about 1 wt % to about 3 wt % acetic acid, propionic acid, or a mixture thereof;
about 3 wt % to about 10 wt % hydrogen peroxide;
about 400 ppm to about 1700 ppm peracetic acid, perpropionic acid or both;
about 0.1 wt % to about 0.3 wt % 1-hydroxyethylidene-1,1,-diphosphonic acid.
